(19) United States
(12) Patent Application Publication
(10) Pub. No.: US 2004/0006431 A1
(43) Pub. Date: Jan. 8, 2004

(54) System, method and computer software product for grid placement, alignment and analysis of images of biological probe arrays

(75) Inventors: Daniel M. Bartell, San Carlos, CA (US); Xiaojun Di, Sunnyvale, CA (US); Wei-Min Liu, Indianapolis, IN (US); Shantanu V. Kaushikkar, San Jose, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, Inc., a Corporation Organized under the laws of Delaware

(21) Appl. No.: 10/391,882

(22) Filed: Mar. 19, 2003

Related U.S. Application Data
(60) Provisional application No. 60/367,146, filed on Mar. 21, 2002.
(60) Provisional application No. 60/393,926, filed on Jul. 3, 2002.
(60) Provisional application No. 60/423,115, filed on Nov. 1, 2002.
(60) Provisional application No. 60/423,911, filed on Nov. 5, 2002.

(51) Int. Cl. G06F 19/00; G01N 33/48; G01N 33/50

(52) U.S. Cl. 702/19

(57) ABSTRACT

A system is described that associates grids with a probe array image based upon a positional placement of one or more control features, aligns the grids with one or more pixels based upon a metric value determined by one or more characteristics of the one or more pixels, and generates cell intensity values. The system may also associate grids with a probe array image based upon a positional placement of one or more control features, determine a ratio of pixel intensity values associated with areas of the grid and adjust the positional association of the grid based upon the determined ratio. The system may also associate grids with a probe array image based upon a positional placement of one or more control features and generate cell intensity values based upon weighted pixel intensity values associated with areas of the grid.

### RELATED APPLICATIONS

The present application claims priority from Provisional Patent Application Serial No. 60/367,146, titled “Image Processing”, filed Mar. 21, 2002; Provisional Patent Application Serial No. 60/393,926, titled “System and Method for Processing Images From Biological Probe Arrays”, filed Jul. 3, 2002; Provisional Patent Application Serial No. 60/423,115, titled “System and Method for Local Grid Adjustment on Images of Biological Robe Arrays”, filed Nov. 1, 2002; and Provisional Patent Application Serial No. 60/423,911, titled “System and Method for Local Grid Adjustment on Images of Biological Robe Arrays”, filed Nov. 5, 2002, all of which are hereby incorporated herein by reference in their entireties for all purposes.

### FIELD OF THE INVENTION

The present invention relates to systems and methods for processing images generated by scanning of arrays of biological materials. The methods include aligning one or more grid patterns to an image of a probe array and determining a value for each image area bounded by an aligned grid.

### BACKGROUND

Synthesized nucleic acid probe arrays, such as Affymetrix® GeneChip® probe arrays, and spotted probe arrays, have been used to generate unprecedented amounts of information about biological systems. For example, the GeneChip® Human Genome U133 Set (HG-U133A and HG-U133B) available from Affymetrix, Inc. of Santa Clara, Calif., is comprised of two microarrays containing over 1,000,000 unique oligonucleotide features covering more than 39,000 transcript variants that represent more than 33,000 human genes. Analysis of expression data from such microarrays may lead to the development of new drugs and new diagnostic tools.

### SUMMARY OF THE INVENTION

The expanding use of microarray technology is one of the forces driving the development of bioinformatics. In particular, microarrays and associated instrumentation and computer systems have been developed for rapid and large-scale collection of data about the expression of genes or expressed sequence tags (EST's) in tissue samples.

Microarray technology and associated instrumentation and computer systems employ a variety of methods to obtain the accurate data from microarray experiments. Researchers are in need of increasingly accurate data generated by microarray technologies. One step in obtaining and analyzing data from microarray experiments may include determining the intensity of sets of probes on an array in one or more scanned images. The intensity typically represents the hybridization of experiment samples to the sets of probes. Synthesized probe arrays may be typically manufactured using photolithography to place identical oligonucleotide probes in rectangular patterns on a base or substrate and the areas containing identical probes are typically referred to as cells. Additionally, spotted probe arrays may be employed in microarray experiments and may be produced in numerous embodiments including embodiments substantially similar to synthesized probe arrays, using various methods, as described below. To determine the intensity of a probe feature, it may be desirable to divide the scanned image into parts representing individual cells. This may be accomplished by processing the scanned image, for example, by placing and/or aligning one or more grids on the scanned image and determining the intensity of pixels comprising individual cells. A strong need exists in the art to make the process of obtaining and analyzing scanned images accurate and reliable.

Systems, methods, and products are described herein to address these and other needs. Various alternatives, modifications and equivalents are possible.

A system is described comprising a grid associater to associate one or more grids with a probe array image based upon a positional placement of one or more control features, a grid aligner to align the grids with pixels of the control features based upon a metric value determined by the characteristics of the pixels, and a cell intensity data generator to generate cell intensity values. A method is also described comprising the acts of associating one or more grids with a probe array image based upon a positional placement of one or more control features, aligning the grids with pixels of the control features based upon a metric value determined by the characteristics of the pixels, and generating cell intensity values.

In another embodiment, a system is described comprising a grid associater to associate one or more grids with a probe array image based upon a positional placement of one or more control features, a grid data calculator to determine a ratio of two sets of pixel intensity values of areas defined by one of the grids, and a grid position adjuster to adjust the positional association of the grid based upon the determined ratio. A method is also described comprising the acts of associating one or more grids with a probe array image based upon a positional placement of one or more control features, determining a ratio of two sets of pixel intensity values of areas defined by one of the grids, and adjusting the positional association of the grid based upon the determined ratio.

In yet another embodiment, a system is described comprising a grid associater to associate one or more grids with a probe array image based upon a positional placement of one or more control features and a cell intensity data generator to generate cell intensity values based upon weighted pixel intensity values associated with an area defined by one of the grids. A method is also described comprising the acts of associating one or more grids with a probe array image based upon a positional placement of one or more control features and generating cell intensity values based upon weighted pixel intensity values associated with an area defined by one of the grids.

The above implementations are not necessarily inclusive or exclusive of each other and may be combined in any manner that is non-conflicting and otherwise possible, whether they are presented in association with a same, or a different, aspect or implementation. The description of one embodiment or implementation is not intended to be limiting with respect to other embodiments or implementations. Also, any one or more function, step, operation, or technique described elsewhere in this specification may, in alternative embodiments or implementations, be combined with any one or more function, step, operation, or technique described in the summary. Thus, the above embodiments or implementations are illustrative rather than limiting.

### BRIEF DESCRIPTION OF THE DRAWINGS

The above and further advantages will be more clearly appreciated from the following detailed description when taken in conjunction with the accompanying drawings. In the drawings, like reference numerals indicate like structures or method steps and the leftmost digit of a reference numeral indicates the number of the figure in which the referenced element first appears (for example, the element 180 appears first in FIG. 1). In functional block diagrams, rectangles generally indicate functional elements, parallelograms generally indicate data, rectangles with curved sides generally indicate stored data, rectangles with a pair of double borders generally indicate predefined functional elements, and keystone shapes generally indicate manual operations. In method flow charts, rectangles generally indicate method steps and diamond shapes generally indicate decision elements. All of these conventions, however, are intended to be typical or illustrative, rather than limiting.

FIG. 1 is a functional block diagram of one embodiment of an image processing system including a scanner and a computer system on which may be executed computer applications suitable for processing image files and for receiving image data and other files for processing;

FIG. 2 is a functional block diagram of one embodiment of application executable including image processing applications as illustratively stored for execution in system memory of the computer system of FIG. 1;

FIG. 3 is a detailed functional block diagram of one embodiment of a grid aligner of FIG. 2, comprising image processing applications;

FIG. 4A is a simplified graphical representation of one embodiment of one or more control features of a probe array images including a grid comprising a plurality of grid lines;

FIG. 4B is a simplified graphical representation of one embodiment of the control features of FIG. 4A spatially arranged in a variety of positions on a probe array image;

FIG. 5 is a simplified graphical representation of the illustrative control features of FIGS. 4A and 4B including a grid comprising a plurality of aligned grid lines that define the boundaries of a plurality of cells;

FIG. 6 is a simplified graphical representation of a plurality of image pixels included in one of the plurality of cells of FIG. 5;

FIG. 7A is a simplified graphical representation of a possible misalignment of the aligned grid lines of FIG. 4A;

FIG. 7B is a simplified graphical representation of a probe array image, before grid alignment, including a grid comprising a plurality of grid lines that define the boundaries of a plurality of cells highlighting the misaligned cells;

FIG. 7C is a simplified graphical representation of the probe array image of

FIG. 7B, after grid alignment, including a grid comprising a plurality of grid lines that define the boundaries of a plurality of cells highlighting the misaligned cells;

FIG. 8 is a simplified graphical representation of the placement of a grid of FIG. 4A comprising a plurality of grid lines that define the boundary of a cell and corresponding image pixels including a fractional portion of a plurality of the image pixels encompassed by a cell; and

FIG. 9 is a functional block diagram of one embodiment of a method for analysis of probe array images by image processing applications of FIG. 2.

### DETAILED DESCRIPTION

The present invention has many preferred embodiments that, in some instances, may include material incorporated from patents, applications and other references for details known to those of the art. When a patent or patent application is referred to below, it should be understood that it is incorporated by reference in its entirety for all purposes.

As used in this application, the singular form “a,” “an,” and “the” include plural references unless the context clearly dictates otherwise. For example, the term “an agent” includes a plurality of agents, including mixtures thereof. An individual is not limited to a human being but may also be other organisms including but not limited to mammals, plants, bacteria, or cells derived from any of the above.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This principle applies regardless of the breadth of the range.

The practice of the present invention may also employ conventional biology methods, software, and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, and other known devices or media and those that may be developed in the future.

The computer executable instructions may be written in a suitable computer language or combination of several languages. As will be appreciated by one of skill in the art, the present invention may be embodied as a method, data processing system or program products. Accordingly, the present invention may take the form of data analysis systems, methods, analysis software, and so on. Software written according to the present invention typically is to be stored in some form of computer readable medium, such as memory, or CD-ROM, or transmitted over a network, and executed by a processor.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both of which are hereby incorporated by reference for all purposes. Some basic methods for image processing are described in, Lisa Gottesfeld Brown: A Survey of Image Registration Techniques, ACM Computing Surveys 24(4): 325-376 (1992), which is hereby incorporated by reference for all purposes.

Computer software products may be written in any of various suitable programming languages, such as C, C++, FORTRAN and Java (Sun Microsystems). The computer software product may be an independent application with data input and data display modules. Alternatively, the computer software products may be classes that may be instantiated as distributed objects. The computer software products may also be component software such as Java Beans (Sun Microsystems), Enterprise Java Beans (EJB), Microsoft® COM/DCOM, etc. The description below is designed to present various embodiments and not to be construed as limiting in any way.

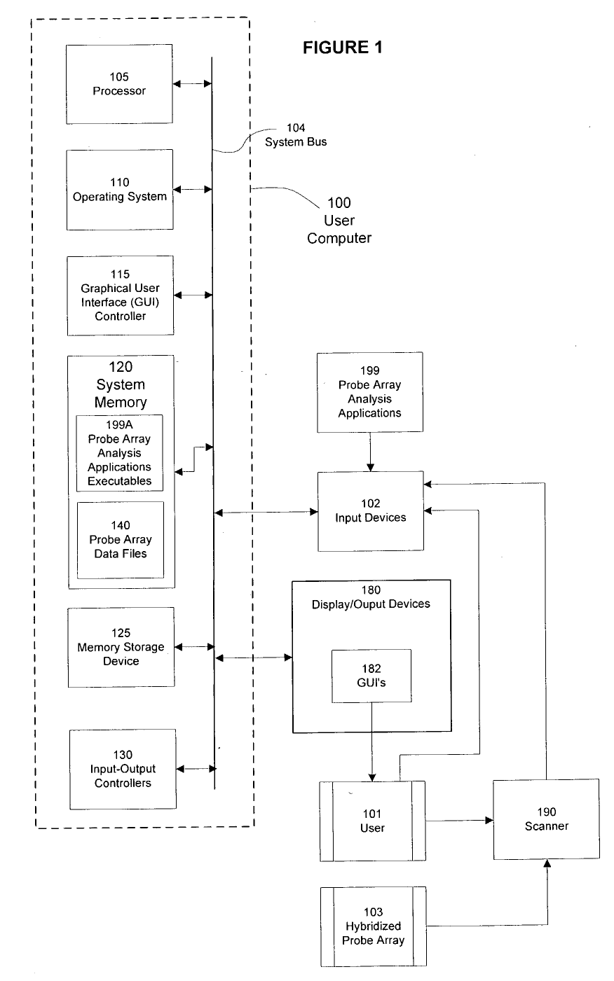

Hybridized Probe Array 103: The example of hybridized probe array 103 provided in FIG. 1 is illustrative only and it will be understood by those of ordinary skill in the related art that numerous variations are possible with respect to providing biological materials for scanning. Various techniques and technologies may be used for synthesizing dense arrays of biological materials on or in a substrate or support. For example, Affymetrix® GeneChip® arrays are synthesized in accordance with techniques sometimes referred to as VLSIPS™ (Very Large Scale Immobilized Polymer Synthesis) technologies. Some aspects of VLSIPS™ and other microarray and polymer (including protein) array manufacturing methods and techniques have been described in U.S. patent Ser. No. 09/536,841, International Publication No. WO 00/58516; U.S. Pat. Nos. 5,143,854, 5,242,974, 5,252,743, 5,324,633, 5,445,934, 5,744,305, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846, 6,022,963, 6,083,697, 6,291,183, 6,309,831 and 6,428,752; and in PCT Applications Nos. PCT/TS99/00730 (International Publication No. WO 99/36760) and PCT/US01/04285, which are all incorporated herein by reference in their entireties for all purposes.

Patents describing specific embodiments of synthesis techniques include U.S. Pat. Nos. 6,486,287, 6,147,205, 6,262,216, 6,310,189, 5,889,165, 5,959,098, and 5,412,087, all hereby incorporated by reference in their entireties for all purposes. Nucleic acid arrays are described in many of the above patents, but the same techniques generally may be applied to polypeptide and other arrays.

Generally speaking, an “array” typically includes a collection of molecules that can be prepared either synthetically or biosynthetically. The molecules in the array may be identical, they may be duplicative, and/or they may be different from each other. The array may assume a variety of formats, e.g., libraries of soluble molecules; libraries of compounds tethered to resin beads, silica chips, or other solid two dimensional or three dimensional supports; and other formats.

The terms “solid support,” “support,” and “substrate” may in some contexts be used interchangeably and may refer to a material or group of materials having a rigid or semi-rigid surface or surfaces. In many embodiments, at least one surface of the solid support will be substantially flat as for e.g. in probe array 103, although in some embodiments it may be desirable to physically separate synthesis regions for different compounds with, for example, wells, raised regions, pins, etched trenches or wells, or other separation members or elements. In some embodiments, the solid support(s) may take the form of beads, resins, gels, microspheres, or other materials and/or geometric configurations providing two or three dimensions for the attachment of probes. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

Generally speaking, a “probe” typically is a molecule that can be recognized by a particular target. To ensure proper interpretation of the term “probe” as used herein, it is noted that contradictory conventions exist in the relevant literature. The word “probe” is used in some contexts to refer not to the biological material that is synthesized on a substrate or deposited on a slide, as described above, but to what is referred to herein as the “target”.

A target is a molecule that has an affinity for a given probe. Targets may be naturally-occurring or man-made molecules. Also, they can be employed in their unaltered state or as aggregates with other species. The samples or targets are processed so that, typically, they are spatially associated with certain probes in the probe array. For example, one or more tagged targets may be distributed over the probe array.

Targets may be attached, covalently or non-covalently, to a binding member, either directly or via a specific binding substance. Examples of targets that can be employed in accordance with this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. Typically, a “probe-target pair” is formed when two macromolecules have combined through molecular recognition to form a complex.

The probes of the arrays in some implementations comprise nucleic acids that are synthesized by methods including the steps of activating regions of a substrate and then contacting the substrate with a selected monomer solution. The term “monomer” generally refers to any member of a set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone. In addition, the terms “biopolymer” and “biological polymer” generally refer to repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer. Related to the term “biopolymer” is the term “biomonomer” that generally refers to a single unit of biopolymer, or a single unit that is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

As used herein, nucleic acids may include any polymer or oligomer of nucleosides or nucleotides (polynucleotides or oligonucleotides) that include pyrimidine and/or purine bases, preferably cytosine, thymine, and uracil, and adenine and guanine, respectively. An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide in accordance with the present invention may be peptide nucleic acid (PNA) in which the constituent bases are joined by peptides bonds rather than phosphodiester linkage, as described in Nielsen et al., Science 254:1497-1500 (1991); Nielsen, Curr. Opin. Biotechnol., 10:71-75 (1999), both of which are hereby incorporated by reference herein. The invention also encompasses situations in which there is a nontraditional base pairing such as Hoogsteen base pairing that has been identified in certain tRNA molecules and postulated to exist in a triple helix. “Polynucleotide” and “oligonucleotide” may be used interchangeably in this application.

Additionally, nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

As noted, a nucleic acid library or array is typically an intentionally created collection of nucleic acids that can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids that can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. Nucleic acid arrays that are useful in the present invention include those that are commercially available from Affymetrix, Inc. of Santa Clara, Calif., under the registered trademark “GeneChip®.” Examples probe arrays may be provided by the website at affymetrix.com.

In some embodiments, a probe may be surface immobilized. Examples of probes that can be investigated in accordance with this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. As non-limiting examples, a probe may refer to a nucleic acid, such as an oligonucleotide, capable of binding to a target nucleic acid of complementary sequence through one or more types of chemical bonds, usually through complementary base pairing, usually through hydrogen bond formation. A probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, or any ligands for detecting its binding partners. Probes of other biological materials, such as peptides or polysaccharides as non-limiting examples, may also be formed. For more details regarding possible implementations, see U.S. Pat. No. 6,156,501, hereby incorporated by reference herein in its entirety for all purposes. When referring to targets or probes as nucleic acids, it should be understood that these are illustrative embodiments that are not to limit the invention in any way.

The term “probe” is used herein to refer to probes such as those synthesized according to the VLSIPS™ technology; the biological materials deposited so as to create spotted arrays; and materials synthesized, deposited, or positioned to form arrays according to other current or future technologies. Thus, microarrays formed in accordance with any of these technologies may be referred to generally and collectively hereafter for convenience as “probe arrays.” Moreover, the term “probe” is not limited to probes immobilized in array format. Rather, the functions and methods described herein may also be employed with respect to other parallel assay devices. For example, these functions and methods may be applied with respect to probes immobilized on or in beads, optical fibers, or other substrates or media. Also, in some cases the sequence and/or composition of the probes may not be known, or may not be fully known.

In accordance with some implementations, some targets hybridize with probes and remain at the probe locations, while non-hybridized targets are washed away. These hybridized targets, with their tags or labels, are thus spatially associated with the probes. The term “hybridization” refers to the process in which two single-stranded polynucleotides bind non-covalently to form a stable double-stranded polynucleotide. The term “hybridization” may also refer to triple-stranded hybridization, which is theoretically possible. The resulting (usually) double-stranded polynucleotide is a “hybrid.” The proportion of the population of polynucleotides that forms stable hybrids is referred to herein as the “degree of hybridization.” Hybridization probes usually are nucleic acids (such as oligonucleotides) capable of binding in a base-specific manner to a complementary strand of nucleic acid. Such probes include peptide nucleic acids, as described in Nielsen et al., Science 254:1497-1500 (1991) or Nielsen Curr. Opin. Biotechnol., 10:71-75 (1999) (both of which are hereby incorporated herein by reference), and other nucleic acid analogs and nucleic acid mimetics. The hybridized probe and target may sometimes be referred to as a probe-target pair. Detection of these pairs can serve a variety of purposes, such as to determine whether a target nucleic acid has a nucleotide sequence identical to or different from a specific reference sequence. See, for example, U.S. Pat. No. 5,837,832, referred to and incorporated above. Other uses include gene expression monitoring and evaluation (see, e.g., U.S. Pat. No. 5,800,992 to Fodor, et al.; U.S. Pat. No. 6,040,138 to Lockhart, et al.; and International App. No. PCT/US98/15151, published as WO99/05323, to Balaban, et al.), genotyping (U.S. Pat. No. 5,856,092 to Dale, et al.), or other detection of nucleic acids. The '992, '138, and '092 patents, and publication WO99/05323, are incorporated by reference herein in their entireties for all purposes.

The present invention also contemplates signal detection of hybridization between probes and targets in certain preferred embodiments. See U.S. Pat. Nos. 5,143,854, 5,578,832; 5,631,734; 5,936,324; 5,981,956; 6,025,601 incorporated above and in U.S. Pat. Nos. 5,834,758, 6,141,096; 6,185,030; 6,201,639; 6,218,803; and 6,225,625, in U.S. Patent application No. 60/364,731 and in PCT Application PCT/US99/06097 (published as WO99/47964), each of which is hereby incorporated by reference in its entirety for all purposes.

A system and method for efficiently synthesizing probe arrays using masks is described in U.S. patent application Ser. No. 09/824,931, filed Apr. 3, 2001, that is hereby incorporated by reference herein in its entirety for all purposes. A system and method for a rapid and flexible microarray manufacturing and online ordering system is described in U.S. Provisional Patent Application Serial No. 60/265,103 filed Jan. 29, 2001, that also is hereby incorporated herein by reference in its entirety for all purposes. Systems and methods for optical photolithography without masks are described in U.S. Pat. No. 6,271,957 and in U.S. patent application Ser. No. 09/683,374 filed Dec. 19, 2001, both of which are hereby incorporated by reference herein in their entireties for all purposes.

As noted, various techniques exist for depositing probes on a substrate or support. For example, “spotted arrays” are commercially fabricated, typically on microscope slides. These arrays consist of liquid spots containing biological material of potentially varying compositions and concentrations. For instance, a spot in the array may include a few strands of short oligonucleotides in a water solution, or it may include a high concentration of long strands of complex proteins. The Affymetrix® 417™ Arrayer and 427™ Arrayer are devices that deposit densely packed arrays of biological materials on microscope slides in accordance with these techniques. Aspects of these and other spot arrayers are described in U.S. Pat. Nos. 6,040,193 and 6,136,269 and in PCT Application No. PCT/US99/00730 (International Publication Number WO 99/36760) incorporated above and in U.S. patent application Ser. No. 09/683,298 hereby incorporated by reference in its entirety for all purposes. Other techniques for generating spotted arrays also exist. For example, U.S. Pat. No. 6,040,193 to Winkler, et al. is directed to processes for dispensing drops to generate spotted arrays. The '193 patent, and U.S. Pat. No. 5,885,837 to Winkler, also describe the use of micro-channels or micro-grooves on a substrate, or on a block placed on a substrate, to synthesize arrays of biological materials. These patents further describe separating reactive regions of a substrate from each other by inert regions and spotting on the reactive regions. The '193 and '837 patents are hereby incorporated by reference in their entireties.

Another technique may include ejecting jets of biological material to form a spotted array. Other implementations of the jetting technique may use devices such as syringes or piezo electric pumps to propel the biological material. It will be understood that the foregoing are non-limiting examples of techniques for synthesizing, depositing, or positioning biological material onto or within a substrate. For example, although a planar array surface is preferred in some implementations of the foregoing, a probe array may be fabricated on a surface of virtually any shape or even a multiplicity of surfaces. Arrays may comprise probes synthesized or deposited on beads, fibers such as fiber optics, glass, silicon, silica or any other appropriate substrate, see U.S. Pat. No. 5,800,992 referred to and incorporated above and U.S. Pat. Nos. 5,770,358, 5,789,162, 5,708,153 and 6,361,947 all of which are hereby incorporated in their entireties for all purposes. Arrays may be packaged in such a manner as to allow for diagnostics or other manipulation in an all inclusive device, see for example, U.S. Pat. Nos. 5,856,174 and 5,922,591 hereby incorporated in their entireties by reference for all purposes.

Also in some implementations, a probe array may consist of a plurality of smaller probe arrays combined onto the same substrate in the manner described above. Such smaller probe arrays may be combined and arranged in any way so long as there is room available upon the substrate. For example a probe array could be constructed from a plurality of miniature probe arrays. The combination of miniature probe arrays could be combined in a variety of combinations to test specific characteristics of a biological sample. Such combinations could reduce the number of individual experiments that user 101 may need to perform resulting in fewer experimental variables and faster results.

Probes typically are able to detect the expression of corresponding genes or ESTs by detecting the presence or abundance of mRNA transcripts present in the target. This detection may, in turn, be accomplished in some implementations by detecting labeled cRNA that is derived from cDNA derived from the mRNA in the target.

The terms “mRNA” and “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In some implementations a group of probes, typically referred to as a probe set, contains sub-sequences in unique regions of the transcripts and does not correspond to a full gene sequence. Further details regarding the design and use of probes and probe sets are provided in PCT Application Serial No. PCT/US 01/02316, filed Jan. 24, 2001 incorporated above; and in U.S. Pat. No. 6,188,783 and in U.S. patent application Ser. No. 09/721,042, filed on Nov. 21, 2000, Ser. No. 09/718,295, filed on November, 21, 2000, Ser. No. 09/745,965, filed on Dec. 21, 2000, and Ser. No. 09/764,324, filed on Jan. 16, 2001, all of which patent and patent applications are hereby incorporated herein by reference in their entireties for all purposes.

The present invention may also make use of various computer program products and software for a variety of purposes, such as probe design, management of data, analysis, and instrument operation. See, U.S. Pat. Nos. 5,593,839, 5,795,716, 5,974,164, 6,090,555, 6,188,783 incorporated above and U.S. Pat. Nos. 5,733,729, 6,066,454, 6,185,561, 6,223,127, 6,229,911 and 6,308,170, hereby incorporated herein in their entireties for all purposes.

FIG. 1 is a functional block diagram illustrating on embodiment of a system that may be suitable for, among other things, analyzing probe arrays that have been hybridized with labeled targets. Representative hybridized probe array 103 of FIG. 1 may include one or more probe arrays of any type, as noted above. Labeled targets in hybridized probe array 103 may be detected using various commercial devices, referred to for convenience hereafter as “scanners.” An illustrative device is shown in FIG. 1 as scanner 190. Generally, scanners may generate an image of one or more targets by detecting fluorescent or other emissions from one or more labels associated with the one or more targets. Additionally, a scanner may detect transmitted, reflected, or scattered radiation. These processes are generally and collectively referred to hereafter for convenience simply as involving the detection of “emissions.” Various detection schemes are employed depending on the type of emissions and other factors. A typical scheme employs optical and other elements to provide excitation light and to selectively collect the emissions. Also generally included are various light-detector systems employing photodiodes, charge-coupled devices, photomultiplier tubes, or similar devices to register the collected emissions. For example, a scanning system for use with a fluorescent label is described in U.S. Pat. No. 5,143,854, incorporated by reference above. Other scanners or scanning systems are described in U.S. Pat. Nos. 5,578,832; 5,631,734; 5,834,758; 5,936,324; 5,981,956; 6,025,601; 6,141,096; 6,185,030; and 6,201,639; in PCT Application PCT/US99/06097 (published as WO99/47964); and in U.S. patent application Ser. No. 09/682,837 filed Oct. 23, 2001, Ser. No. 09/683,216 filed Dec. 3, 2001, and Ser. No. 09/683,217 filed Dec. 3, 2001, Ser. No. 09/683,219 filed Dec. 3, 2001, each of which is hereby incorporated by reference in its entirety for all purposes.

Scanner 190: In the illustrated example of FIG. 1, scanner 190 may provide data representing the intensities (and possibly other characteristics, such as color) of detected emissions, as well as the locations on the substrate where the emissions were detected. The data typically are stored in a memory device, such as system memory 120 of computer 100, in the form of a data file or other data storage form or format. One type of data, image data 222, typically includes intensity and location information corresponding to elemental sub-areas of the scanned substrate. The term “elemental” in this context means that the intensities, and/or other characteristics of the emissions from this area each may be represented by a single value. When displayed as an image for viewing or processing, elemental picture elements, or pixels, often represent this information. Thus, for example, a pixel may have a single value representing the intensity of the elemental sub-area of the substrate from which the emissions were scanned. The pixel may also have another value representing another characteristic, such as color. For instance, a scanned elemental sub-area in which high-intensity emissions were detected may be represented by a pixel having high luminance (hereafter, a “bright” pixel), and low-intensity emissions may be represented by a pixel of low luminance (a “dim” pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular area or sub-area of the substrate may be represented as a red pixel, and emissions of a second wavelength detected at another area or sub-area may be represented by an adjacent blue pixel. Many other display schemes are known. Two non-limiting examples of image data may include data files in the form *.dat or *.tif as generated respectively by Affymetrix® Microarray Suite based on images scanned from GeneChip® arrays, and by Affymetrix® Jaguar™ software based on images scanned from spotted arrays.

User Computer 100: User computer 100, shown in FIG. 1, may be a computing device specially designed and configured to support and execute some or all of the functions of image processing applications 199. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as a Pentium® processor made by Intel Corporation, a SPARC® processor made by Sun Microsystems, or it may be one of other processors that are or will become available. Processor 105 executes operating system 110, which may be, for example, a Windows®-type operating system (such as Windows NT® 4.0 with SP6a) from the Microsoft Corporation; a Unix® or Linux-type operating system available from many vendors; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, or a diskette drive. Such types of memory storage device 125 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUT) controller 115 may comprise any of a variety of known or future software programs for providing graphical input and output interfaces such as GUI 182, between computer 100 and user 101, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

Probe-Array Analysis Applications 199/Probe-Array Analysis Applications Executables 199A: Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided for processing by a computer application that associates the locations where hybridized targets were detected with known locations where probes of known identities were synthesized or deposited. Other methods include tagging individual synthesis or support substrates (such as beads) using chemical, biological, electromagnetic transducers or transmitters, and other identifiers. Information such as the nucleotide or monomer sequence of target DNA or RNA may then be deduced. Techniques for making these deductions are described, for example, in U.S. Pat. No. 5,733,729 and in U.S. Pat. No. 5,837,832, noted and incorporated above.

As mentioned earlier, synthesized probe arrays may be manufactured using photolithography to place identical oligonucleotide probes in distinct patterns, including rectangular patterns, on a base or substrate, and the areas containing identical probes are typically referred to as probe features or “cells”. Furthermore, the term “cell” may be used descriptively to refer to the spots in case of spotted arrays. In the present context the term “cell” may be used broadly and descriptively to refer to an individual unit area, or grid element, bounded by grid lines. In one preferred embodiment, the grid elements or “cells” may have similar characteristics, including size and/or shape, as the areas containing identical probes on a synthesized probe array or the spots on a spotted array.

A variety of computer software applications are commercially available for controlling scanners (and other instruments related to the hybridization process, such as hybridization chambers), and for acquiring and processing the image files provided by the scanners. Examples are the Jaguar™ application from Affymetrix, Inc., aspects of which are described in PCT Application PCT/US 01/26390 and in U.S. patent applications, Ser. Nos. U.S.20020047853, 09/682,071, 09/682,074, and 09/682,076, all of which are hereby incorporated herein by reference in their entireties for all purposes, and the Microarray Suite application from Affymetrix, aspects of which are described in U.S. Provisional Patent Applications, Serial Nos. 60/220,587, 60/220,645, 60/226,999 and 60/312,906, U.S. patent application Ser. No. 10/219,882, all of which are also hereby incorporated herein by reference in their entireties for all purposes. Aspects of software applications for acquiring and processing the image files provided by the scanners are also described in U.S. Patent Application No. 60/408,848 and in U.S. Patent Application No. 60/442,684, filed January, 24, 2003, all of which are hereby incorporated herein by reference in their entireties for all purposes. For example, image data in an image data file may be operated upon to generate intermediate results such as so-called cell intensity files (*.cel) and chip files (*.chp), generated by Microarray Suite or spot files (*.spt) generated by Jaguar™ software. Additionally, the processing of images produced by scanning probe arrays may employ placing and aligning a grid over an image and determining the intensity of signals within the cells of the grid.

For convenience, the terms “file” or “data structure” may be used herein to refer to the organization of data, or the data itself generated or used by executables 199A and executable counterparts of other applications. However, it will be understood that any of a variety of alternative techniques known in the relevant art for storing, conveying, and/or manipulating data may be employed, and that the terms “file” and “data structure” therefore are to be interpreted broadly.

The intensity of signals is typically a measure of the abundance of tagged cRNAs present in the target that hybridized to the corresponding probe. Many such cRNAs may be present in each probe, as a probe on a GeneChip® probe array may include, for example, millions of oligonucleotides designed to detect the cRNAs. The resulting data stored in the chip file may include degrees of hybridization, absolute and/or differential (over two or more experiments) expression, genotype comparisons, detection of polymorphisms and mutations, and other analytical results. In another example, in which executables 199A include image data from a spotted probe array, the resulting spot file includes the intensities of labeled targets that hybridized to probes in the array. Further details regarding cell files, chip files, and spot files are provided in U.S. Provisional Patent Application Nos. 60/220,645, 60/220,587, and 60/226,999, incorporated by reference above.

In the present example, in which executables 199A may include aspects of Affymetrix® Microarray Suite, the chip file is derived from analysis of the cell file combined in some cases with information derived from library files (not shown) that specify details regarding the sequences and locations of probes and controls. Laboratory or experimental data may also be provided to the software for inclusion in the chip file. For example, an experimenter and/or automated data input devices or programs (not shown) may provide data related to the design or conduct of experiments. As a non-limiting example related to the processing of an Affymetrix® GeneChip® probe array, the experimenter may specify an Affymetrix catalog or custom chip type (e.g., Human Genome U95Av2 chip) either by selecting from a predetermined list presented by Microarray Suite or by scanning a bar code related to a chip to read its type. Microarray Suite may associate the chip type with various scanning parameters stored in data tables such as, for instance, the area of the chip that is to be scanned, the location of chrome borders on the chip that may be used for auto-focusing, the wavelength or intensity of laser light to be used in reading the chip, and so on. Other experimental or laboratory data may include, for example, the name of the experimenter, the dates on which various experiments were conducted, the equipment used, the types of fluorescent dyes used as labels, protocols followed, and numerous other attributes of experiments. As noted, executables 199A may apply some of this data in the generation of intermediate results. For example, information about the dyes may be incorporated into determinations of relative expression. Other data, such as the name of the experimenter, may be processed by executables 199A or may simply be preserved and stored in files or other data structures. Any of these data may be provided, for example over a network, to a laboratory information management server computer, configured to manage information from large numbers of experiments. As will be appreciated by those skilled in the relevant art, the preceding and following descriptions of files generated by executables 199A are illustrative only, and the data described, and other data, may be processed, combined, arranged, and/or presented in many other ways.

The processed image files produced by these applications often are further processed to extract additional data. In particular, data-mining software applications often are used for supplemental identification and analysis of biologically interesting patterns or degrees of hybridization of probe sets. An example of a software application of this type is the Affymetrix® Data Mining Tool, described in U.S. Provisional Patent Applications, Serial Nos. 60/274,986 and 60/312,256, and U.S. patent application Ser. No. 09/683,980 each of which is hereby incorporated herein by reference in their entireties for all purposes. Software applications also are available for storing and managing the enormous amounts of data that often are generated by probe-array experiments and by the image-processing and data-mining software noted above. An example of these data-management software applications is the Affymetrix® Laboratory Information Management System (LIMS), aspects of which are described in U.S. Provisional Patent Applications, Serial Nos. 60/220,587 and 60/220,645, incorporated above and in U.S. patent application Ser. No. 09/682,098 hereby incorporated by reference herein in its entirety for all purposes. In addition, various proprietary databases accessed by database management software, such as the Affymetrix® EASI (Expression Analysis Sequence Information) database and database software, provide researchers with associations between probe sets and gene or EST identifiers.

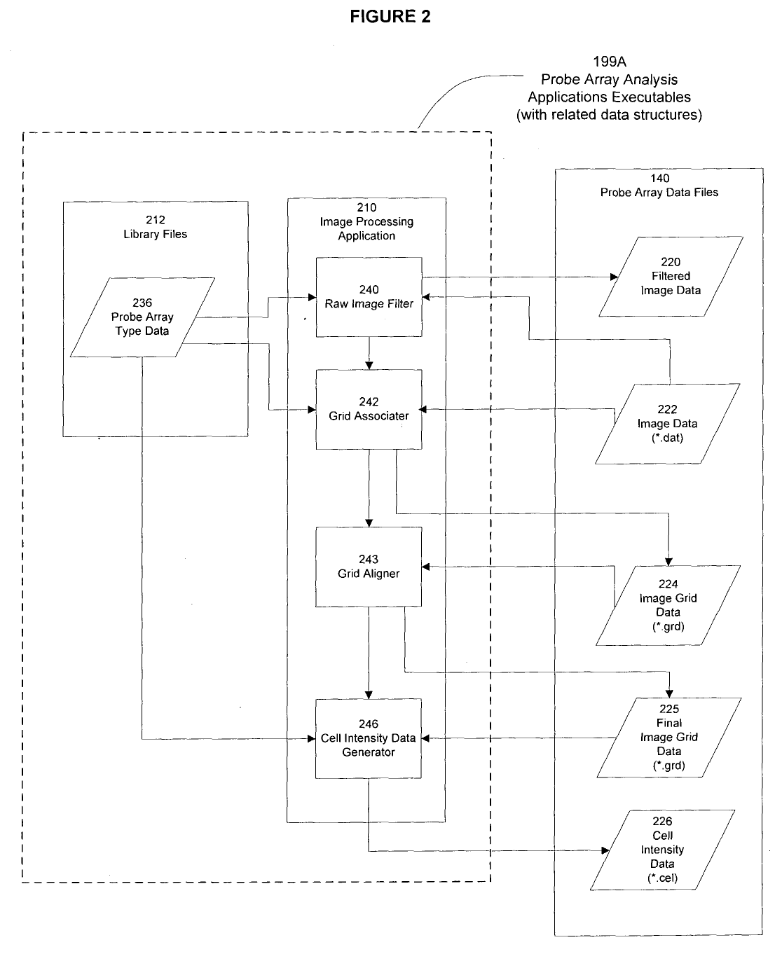

For convenience of reference, these types of computer software applications (i.e., for acquiring and processing image files, data mining, data management, and various database and other applications related to probe-array analysis) are generally and collectively represented in FIG. 2 as probe-array analysis applications/executables 199A.

As will be appreciated by those skilled in the relevant art, it is not necessary that applications/executables 199A be stored on and/or executed from computer 100; rather, some or all of applications/executables 199A may be stored on and/or executed from an applications server or other computer platform to which computer 100 is connected in a network. For example, it may be particularly advantageous for applications involving the manipulation of large databases, such as Affymetrix® LIMS or Affymetrix® Data Mining Tool (DMT), to be executed from a database server. Alternatively, LIMS, DMT, and/or other applications may be executed from computer 100, but some or all of the databases upon which those applications operate may be stored for common access on a server (perhaps together with a database management program, such as the Oracle® 8.0.5 database management system from Oracle Corporation). Such networked arrangements may be implemented in accordance with known techniques using commercially available hardware and software, such as those available for implementing a local-area network or wide-area network. A local network could be represented by the connection of user computer 100 to a user database server (and to a user-side Internet client, which may be the same computer) via a network cable. Similarly, scanner 190 (or multiple scanners) may be made available to a network of users over the network cable both for purposes of controlling scanner 190 and for receiving data input from it.

Image Processing Application 210: Some implementations of probe array analysis executables 199A may include image processing application 210 as illustrated in FIG. 2. It may be desirable to process images produced from scanned hybridized probe arrays using a variety of methods.

Several implementations include placing/associating and aligning a grid over an image and determining the intensity of signals within the cells of the grid. FIG. 2 is an illustrative example of one possible implementation of image processing application 210 that includes elements for grid placement, grid alignment and determination of cell intensity, illustrated as grid associater 242, grid aligner 243 and cell intensity data generator 246.

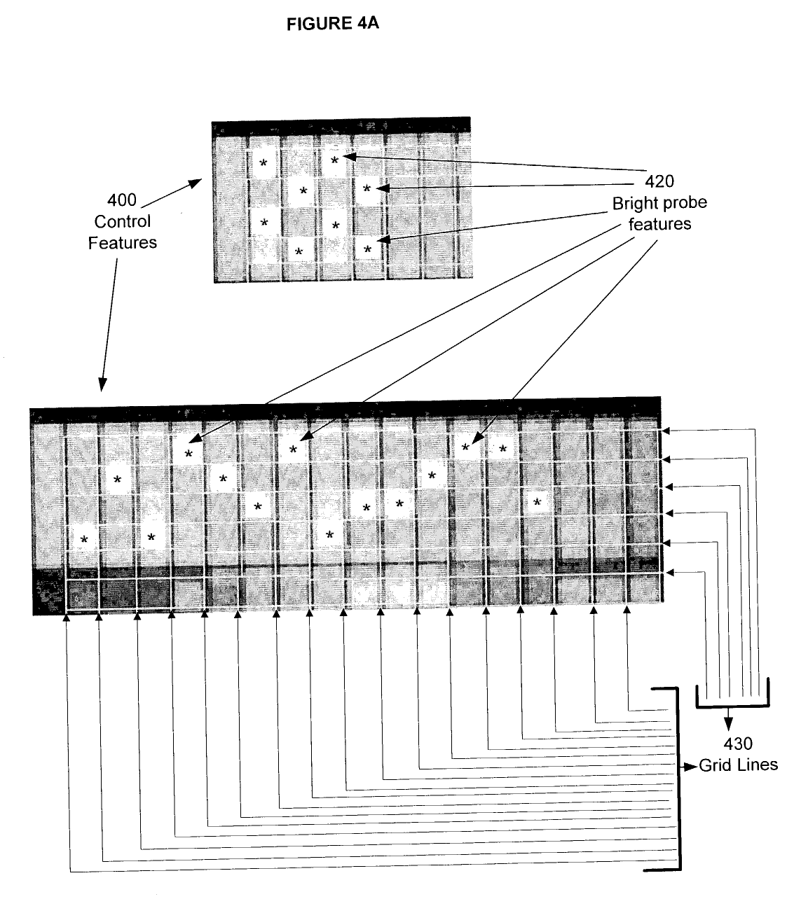

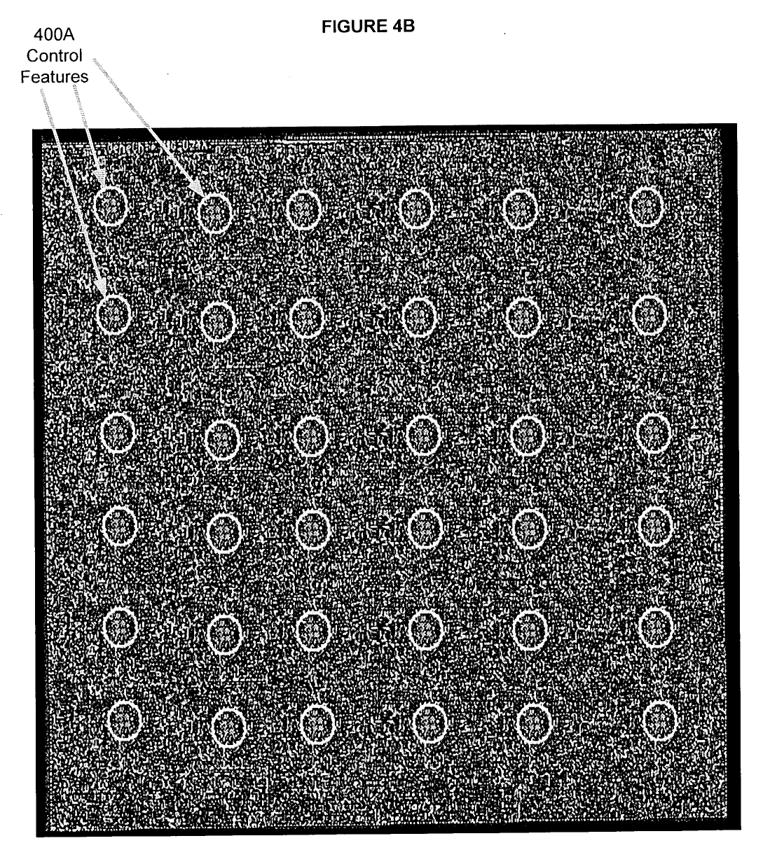

Some implementations may include the use of one or more control features to determine one or more positions for grid placement. Presented in FIG. 4A is an illustrative example of control features 400, also referred to as “fiducial feature” in some embodiments that may include one or more patterns such as, for instance, a pattern of chrome features and/or one or more arrangements of probes on the probe array. The chrome features and/or the arrangement of probes may include one or more specific patterns that a computer or user could easily recognize. As a non-limiting example, crosshair shapes/patterns of chrome may be placed on a probe array at one or more predetermined locations for use as reference or anchor points.

In another embodiment, control features 400 may include serrated chrome lines placed at specific locations, e.g. around the area where probes are attached, the serrations serving as reference points. In the same or another embodiment, one or more control features may be composed of various materials other than chrome, including one or more materials comprising the probes, fluorescent labels and/or array substrate. In a non-limiting example, one or more probes bound to fluorescent labels, located at predetermined locations, may serve as reference points. In yet another implementation one or more control features may comprise a checkerboard or other pattern that may include a pattern of hybridized probe features such as, for instance, probe features that may be sensitive to target sequences in a sample. The target sequence may include oligonucleotide sequences that a user may add to an experimental sample for what is commonly referred to as a control for the hybridization protocol. In a non-limiting example one or more control features may be located at each corner of the probe array image, such as, for instance, control features shown in FIG. 4A and further described below.

In the same or another embodiment, the control features may be distributed over the probe containing area, also referred to as the “active area”, of a probe array, as illustrated in FIG. 4B and further described below. Other examples pertaining to incorporating and using fiducial features on probe arrays may be found in U.S. Provisional Patent Application Serial Nos. 60/364,731, 60/396,457, 60/435,178, 09/683,216, 09/683,217, 09/683,219, and 60/443,402, each of which is hereby incorporated by reference in its entirety for all purposes.

The term “placement”, as used in “grid placement” typically implies an act of associating/placing a grid on an image obtained by scanning a probe array. Similarly, the term “alignment”, as used in “global grid alignment” or in “local grid alignment” or in “grid alignment”, typically implies an act of adjusting one or more characteristics (e.g. position) of a grid or a part of a grid, associated/placed on a scanned image of a probe array, in order to place the grid or a part of the grid within one pixel or fractional pixel of optimum position around one or more features. It must also be mentioned that in certain instances a grid may be inappropriately placed or aligned with one or more features not intended for use in grid placement or alignment methods. In this context the term “registration”, as used in “grid registration” implies accurate placement or alignment of the grid around one or more features which are supposed to be used for placement or alignment. It will be understood that a “grid” typically may be a construct embodied in data or classification/arrangement of data, rather than in a physical manifestation.

One or more of control features 400 may be located at one or more predetermined locations on a probe array for use as reference or anchor points. The predetermined locations of control features 400 may be stored in one or more data files or databases that for instance could include a library or other type of file describing the type of probe array. A library or other type of file may be stored remotely on one or more servers, or locally as illustrated in FIG. 2 as library files 212.

Placement and alignment of one or more grids may be accomplished in a variety of methods as described below. One embodiment may include implementing an initial step of grid placement using control features 400 followed by grid alignment. In the present embodiment one or more probe array images may be represented by probe array image data 222 that may include emission intensity data from hybridized probe arrays 103 acquired by scanner 190. Illustrative elements of application 210 such as raw image filter 240, grid associater 242, grid aligner 243, and cell intensity data generator 246, may implement the methods of grid placement/association and/or grid alignment as described in detail below.

In the illustrated implementation filter 240 may perform a set of calculations for each pixel of image data 222. For example, the calculations may include a test of whether a pixel represents an element of control feature 400. For every pixel of image data 222, filter 240 selects a plurality of additional pixels at predetermined positions in relation to the selected test pixel and includes this data in filtered image data 220. Filter 240 may accomplish this by employing numerous methods including those described in U.S. Pat. No. 6,090,555, and Provisional Patent Application Serial No. 60/423,911, titled “System and Method for Local Grid Adjustment on Images of Biological Robe Arrays”, filed Nov. 5, 2002, incorporated above.

Continuing the example above, filter 240 may determine whether a test pixel represents an element of control features 400 by a comparison of an expected positional arrangement and intensity values of the plurality of additional pixels in relation to the test pixel to calculated arrangements and intensity values. To determine if the test pixel is a part of a control feature 400, filter 240 may calculate the average emission intensity of the selected additional pixels, and compare the values to expected average emission intensity values corresponding to elements of control feature 400. A control feature 400 could for instance comprise one or more bright probe features 420, marked by “*” in FIG. 4A. Furthermore, raw image filter 240 may calculate an expected positional arrangement of the selected additional pixels based, at least in part, upon position data of control feature 400 from probe array type data 236 and determine an expected emission intensity value associated with each of the one or more control features. Additionally, raw image filter 240 may assemble all of the filtered pixel values and produce one or more filtered probe array images as represented by filtered image data 220 of FIG. 2. Other methods for determining pixel locations and boundaries of probe features and/or control features are described in further detail in U.S. Patent Application, Serial No. U.S.20020047853, incorporated above.

Filtered image data 220 may be stored in probe array data files 140 and/or forwarded by raw image filter 240 to grid associater 242. Grid associater 242 may receive filtered image data 220 and/or image data 222 to perform a multi-step process for associating the grid with a probe array image or grid placement. In one embodiment of the invention, associater 242 uses data 220 to determine a plurality of pixel positions for grid placement, such as, for instance one or more pixel positions associated with elements of control features 400 located at the corners of the probe array image of image data 220. Associater 242 may place a grid on image data 222 using pixel positions selected by filter 240 above to anchor or fix the grid for placement.

For example, a grid may comprise horizontal and vertical orientations of grid lines 430 as shown in FIG. 4A. Grid lines 430 limit or bound the cells or grid elements of a grid placed over a probe array image. The shape and size of cells bounded by grid lines 430 typically correspond to the actual size and shape of the probe features present on hybridized probe array 103.

However, as will be appreciated by those of ordinary skill in the art, a grid may be represented in numerous other configurations and patterns, with numerous other characteristics than those described here, for example, a grid may comprise concentric circles with radial projections, or intersecting or non-intersecting geometric shapes and/or any combinations thereof giving rise to a grid pattern. Furthermore, the grid may comprise cells of various shapes and sizes including but not limited to rectangular, hexagonal and circular shapes. Thus the illustrations and descriptions of a grid or grid pattern in the illustrative examples disclosed here should not be interpreted or construed to be limiting or restrictive in any manner whatsoever.

In one implementation, the pixel positions employed to anchor a grid may be located near the corners of the probe array image so that the global position of an associated grid is correct with respect to the outer edges of the active area of the probe array. For example, to determine the plurality of pixel positions to anchor the grid, associater 242 performs a series of searches of filtered image data 220. Each search may begin at one of the corners of filtered image data 220 and continues towards the center of image data 220 until a bright pixel is found. In the present example, associater 242 uses the pixel position corresponding to each bright pixel at each corner as anchor positions for grid placement. Additionally, probe array type data 236 provides the number of probe features associated with the probe array and thus determines the number of cells of the grid.

In the same or other implementations each cell may correspond to a probe feature of a particular implementation of hybridized probe array 103, but many variations are possible. Further details and embodiments are also described in Provisional Patent Application Serial No. 60/423,911, titled “System and Method for Local Grid Adjustment on Images of Biological Probe Arrays”, filed Nov. 5, 2002, incorporated above. Associater 242 produces image grid data 224 that may include an intensity value corresponding to each pixel of the probe array image, and additionally may be stored in probe array data files 140 of FIG. 2.

In some instances, the quality and accuracy of the positional placement of the grid with respect to probe features near the center of the image may deteriorate. The source of error of the globally positioned grid may include distortion of the probe array image introduced by the scanner 190. Such errors in grid placement could lead to inaccurate measurements of cell intensity and may be ameliorated by optimally aligning the grid with the probe array image for example by grid aligner 243 as described below.

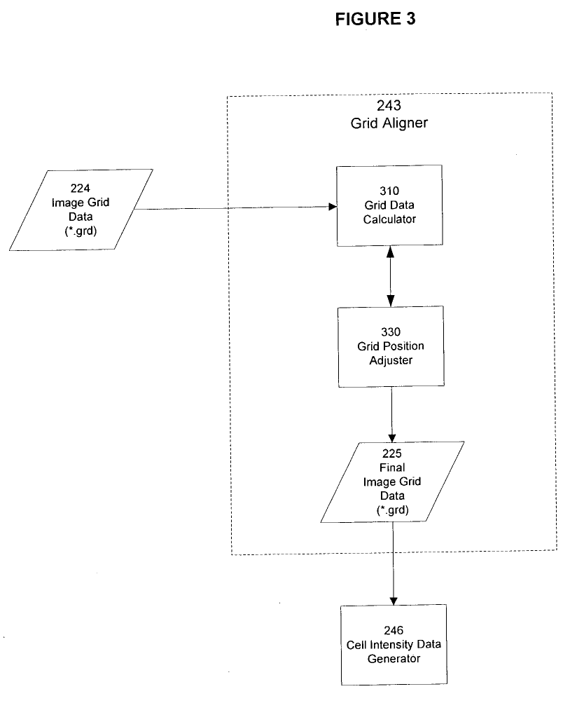

Grid aligner 243, as illustrated in FIG. 3, includes grid data calculator 310 and grid position adjuster 330. Calculator 310 is capable of calculating an intensity value associated with each of the cells that a grid may comprise. In one embodiment of the invention, aligner 243 divides the grid pattern into sub grids that may comprise one or more cells. In the same or other embodiments the globally positioned grid may comprise one or more or a combination of sub grids. Probe array type data 236 may provide the size, number, and coordinate positions of each of the sub grids. Probe array type data 236 may vary according to a particular implementation of probe array 103, or alternatively may be user definable. Grid position adjuster 330 is capable of changing various characteristics of a grid including but not limited to, the size of the cells and the position of one or more sub grids, based upon the data provided by the calculator 310.

In one embodiment, calculator 310 may provide adjuster 330 with the average intensity value corresponding to each of the one or more cells, calculated from the intensity values corresponding to each of pixels encompassed by each cell. Adjuster 330 may adjust the position of the sub grid based on the average intensity value and may return the image data with the adjusted sub grid to calculator 310 for recalculation of the average intensity values. For example, adjuster 330 may adjust the position of a sub grid by moving or relocating the sub grid over the probe array image in one or more directions using one or more predefined or user selectable criteria, for example, until the average intensities of one or more cells of the sub grid are suitably close to predefined or user selectable intensities.

In one embodiment, the grid pattern may comprise sub grids so that associater 242 associates one or more sub grids with one or more control feature, as for example the control features 400A illustrated in FIG. 4B. It must be noted here that control features 400A may be similar to control features 400, but are labeled as 400A for illustrative purposes only.

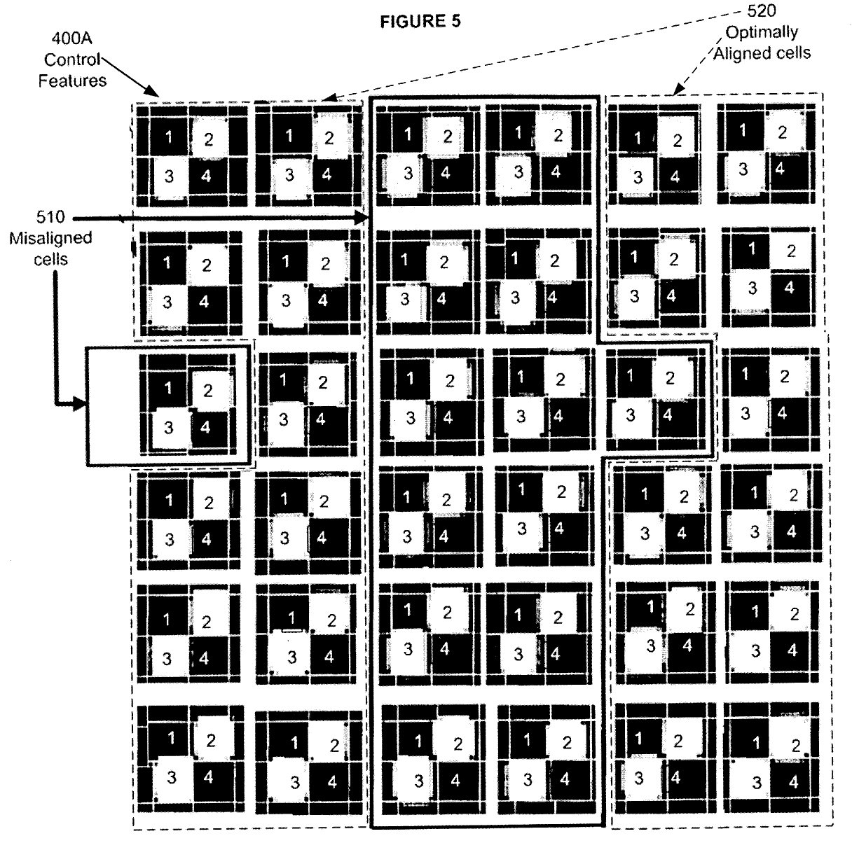

FIG. 4B illustrates a possible distribution of control features 400A over a probe array image. The control features 400A are too small to be delineated in FIG. 4B, therefore, in this illustrative example, the locations of 36 such control features 400A are highlighted by encircling them with white lines for the sake of clarity. The same 36 control features 400A are magnified and shown together in FIG. 5. As shown in FIG. 4B, control features 400A may be distributed regularly and/or evenly over the probe array image. Additionally, in this illustrative example, each one of control features 400A corresponds to a sub grid comprising four cells numbered as 1, 2, 3 and 4. However, this need not be so in every embodiment, and as will be appreciated by those of ordinary skill in the art, numerous other variations of characteristics including but not limited to appearance, size, shape, number, geometry and arrangement may be employed for the same purpose. Thus the illustrations and descriptions of features 400 and 400A are illustrative only and should not be interpreted or construed to be limiting or restrictive in any manner whatsoever.

In the same or other embodiments, the probe features or cells of control features 400A may comprise probe sequences sensitive to target sequences in a sample. The target sequence may include oligonucleotide sequences added to an experimental sample for what is commonly referred to as a “control” for the hybridization protocol. This typically ensures that the control features will be hybridized in a predetermined manner. In this illustrative example, cells 2 and 3 of features 400A appear bright as compared to cells 1 and 4.

Aligner 230 may align one or more grids or sub-grids, by employing one or more of the numerous methods described below.

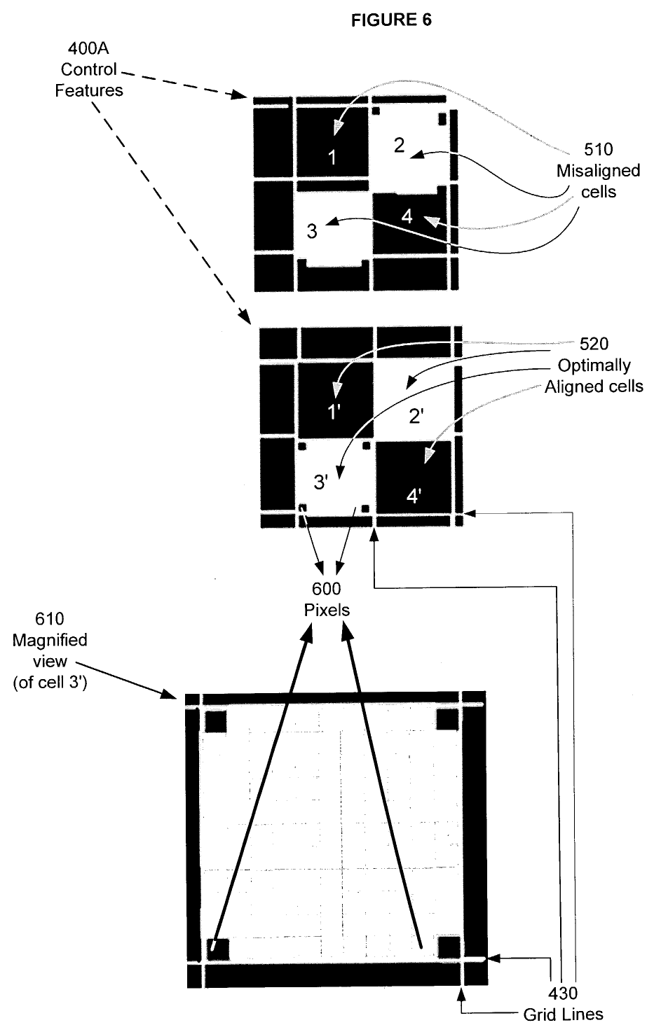

As shown illustratively in FIG. 5, the grid may be misaligned in numerous ways with respect to the features of probe array 103. Control features 400 may be misaligned with respect to one or more cells bounded by grid lines 430, labeled as misaligned cells 510. Alternatively, there may be other cells in the same probe array image aligned optimally with all other control features, labeled as optimally aligned cells 520. This difference is illustrated in greater detail in FIG. 6.

FIG. 6 shows illustratively one of the many possible examples of misaligned cells 510 and optimally aligned cells 520. In this illustrative example, a plurality of misaligned cells 510 are misaligned with a control feature 400A and are labeled individually as 1,2,3 and 4, similarly a plurality of optimally aligned cells 520 are optimally aligned with a control feature 400A and are, labeled individually as 1′,2′,3′ and 4′. Cell 3′ of optimally aligned cells 520 is shown in a magnified view 610 to illustrate further details.

Typically each of the misaligned cells 510 and optimally aligned cells 520 comprises one or more pixels 600. As described above, a pixel is an elemental picture element. In this illustrative example, cell 3′ may include an 11×11 array pattern of pixels totaling 121 pixels, illustrated here as delineated by discontinuous lines in the magnified view 610. For example, four of the pixels 600, located at the four corners of cell 3′ are dark when compared to the other pixels of cell 3′. In FIG. 6, cells 1′ and 4′ are completely comprised of dark pixels and no bright pixels, whereas cell 2′ is completely comprised of bright pixels and no dark pixels. However, this need not be so in every embodiment, and as will be appreciated by those of ordinary skill in the art, one or more cells in the same or other embodiments may comprise pixels of numerous other variations of characteristics, including but not limited to appearance, size, shape, number, geometry and arrangement of the pixels. Thus the above mentioned illustrations and their descriptions are illustrative only and should not be interpreted or construed to be limiting or restrictive in any manner whatsoever.

In some implementations, grid data calculator 310 may be capable of calculating, an intensity value corresponding to each of the cells of a grid or sub grid. Additionally, grid position adjuster 330, may be capable of changing various characteristics of a grid or sub grid including but not limited to, the size of the cells and the position of the grid or sub grid, based at least in part upon the data provided by the calculator 310.

In an illustrative example, control features 400A may be spread over a probe array image in N×M array pattern of N rows and M columns. For further processing calculator 310 may represent the co-ordinates of pixels encompassed by the cells of a sub grid corresponding to a control feature 400A, in the following manner:

{(xᵢⱼ, yᵢⱼ)|i=1, . . . , N; j=1, . . . , M}

and the co-ordinates of cells that the sub grid comprises may be represented as:

{(^{nx}ᵢⱼ, nyᵢⱼ)|i=1, . . . , N; j=1, . . . , M}

As will be appreciated by those of ordinary skill in the art, the variables used in the equations described above and hereon, including but not limited to xᵢⱼ, yᵢⱼ, nxᵢⱼ and nyᵢⱼ, are used illustratively and in a non-limiting manner and are to interpreted in the present context as numerical/mathematical/statistical entities as known in the art and should not be interpreted or construed to be limiting or restrictive in any manner whatsoever.

Calculator 310 may calculate a numerical score and assign it to one or more control features 400A by employing the following equation:

$$Score = max ( \frac{Ave ( Cell 1 ) + Ave ( Cell 4 )}{Ave ( Cell 2 ) + Ave ( Cell 3 )} , \frac{Ave ( Cell 2 ) + Ave ( Cell 3 )}{Ave ( Cell 1 ) + Ave ( Cell 4 )}$$

where Ave(Cell1), Ave(Cell2), Ave(Cell3) and Ave(Cell4) represent the average of the intensities of the pixels that the cells 1, 2, 3 and 4 respectively comprise, and max represents the larger or maximal of the value or values calculated by employing the equation. Other statistical measures or techniques may also be used.

Furthermore, grid position adjuster 330 may adjust the positional location of a grid or sub-grid by repositioning or moving the grid or sub-grid with respect to one or more of the N×M control features 400A. Alternatively in the same or other embodiments features 400A may be moved with respect to the grid or sub grid. In yet another embodiment, both the grid or the sub grid and the features 400A may be moved with respect to each other. It must be mentioned here that the grid or sub grid may be repositioned or move in one or more directions by a predefined or user selectable magnitude of movement. In an illustrative example, adjuster 330 may move a grid or sub grid by a magnitude of movement equal to multiple pixel lengths, including fractional pixel lengths, for example, by 1 pixel length or by 1.2 pixel lengths or 2 pixel lengths and so on.

The co-ordinates of pixels that the cells of a grid or sub-grid encompass in the new position and/or location may be represented as:

{(x′ᵢⱼ, y′ᵢⱼ)|i=1, . . . , N; j=1, . . . , M}

The new alignment or location/position of the sub grid may be optimal as based on one or more predefined or user selectable criteria. In a non-limiting illustrative example, these criteria may include comparing the score calculated and assigned to a control feature 400A in one position of the grid or sub-grid to one or more scores calculated and assigned the same control feature 400A corresponding to one or more other positions of the grid or sub-grid and then selecting the position providing a score suitably close to a pre defined or user selectable score. Additional details and methods that may be employed are described in U.S. Patent Application, Serial No. U.S.20020047853, incorporated above.

Furthermore, grid data calculator 310 may calculate δᵢⱼ and εᵢⱼ such that,

δᵢⱼ=x′ᵢⱼ−x_{ij,} where 1≦i≦N and

εᵢⱼ=y′ᵢⱼ−y_{ij,} where 1≦j≦M

As will now be appreciated by those of skill in the art, δᵢⱼ and εᵢⱼ may be referred to as “offsets” of the control features 400A from their initial to their final and/or optimal positions, along the rows and columns respectively.

Furthermore, calculator 310 may calculate the median values or other statistical measures of the offsets represented by δᵢ and εⱼ, along the rows and columns respectively, such that,

67 ᵢ=MEDIAN(δᵢ₁, . . . , δ_{iM}), where 1≦i≦N and

εⱼ=MEDIAN(ε₁ⱼ, . . . , ε_{Nj}), where 1≦j≦M

thus defining two sets of median values, namely X and Y, such that,

X={δ₀δ₁, . . . δ_{N},δ_{N+1}} and

Y={ε₀,ε₁, . . . , ε_{M},ε_{M+1}}

where δ₀=0, δ_{N+1}=0, ε₀=0 and ε_{M+1}=0

Calculator 310 may further calculate the data required for aligning the grid over probe array image based at least in part upon the above calculated data, so that (x, y) represent the co-ordinates of pixels for every cell of a grid or sub-grid and (x′, y′) represents the co-ordinates after optimal alignment of the cells. The co-ordinates (x′, y′) may be calculated as:

$${x}^{′} = x + \frac{( {δ}_{i} - {δ}_{i - 1} ) ( x - {x}_{i - 1 , j} )}{x - ij - {x}_{i - 1 , j}} and$$

$${y}^{′} = y + \frac{( {ϵ}_{j} - {ϵ}_{j - 1} ) ( y - {y}_{i , j - 1} )}{{y}_{ij} - {y}_{i , j , - 1}}$$

Alternatively, in the same or other embodiments, the grid may be divided into smaller regions based at least in part upon the N×M pattern of control features 400A, for example the image may be divided into (N+1)×(M+1) regions and the above calculations performed to align each divided region of the grid with the image.

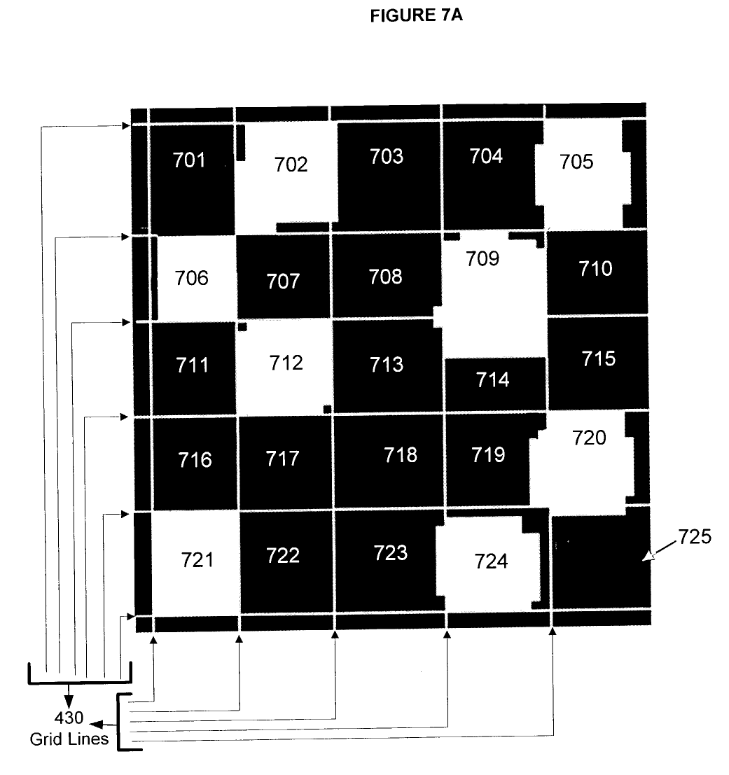

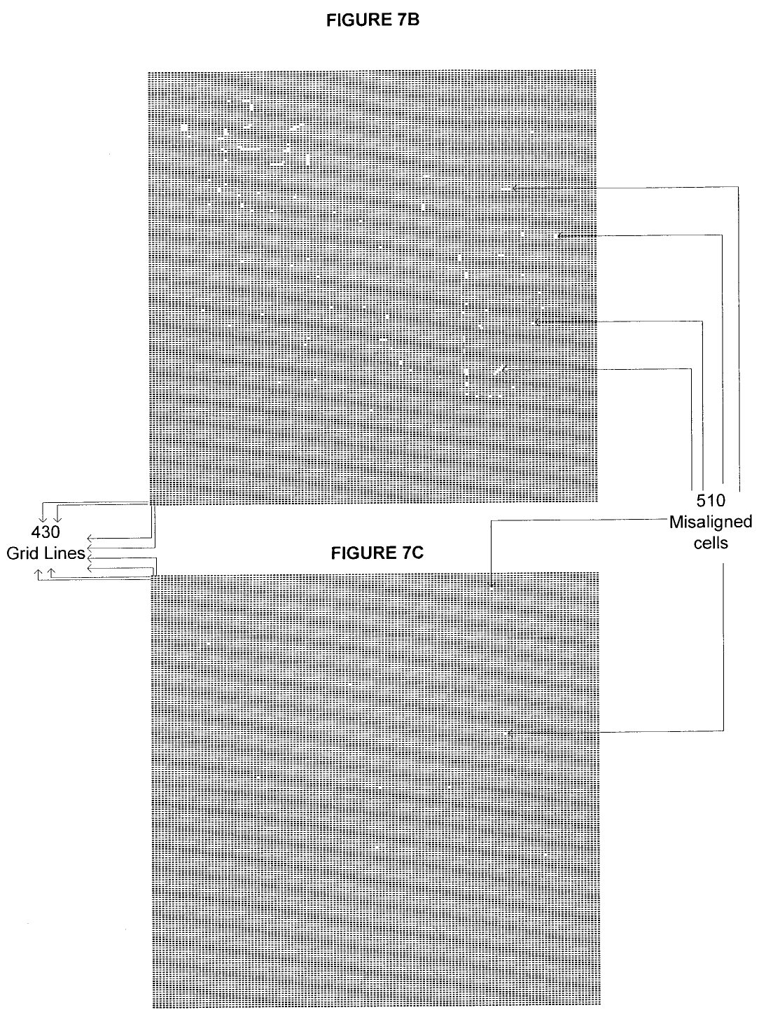

In the same or other embodiments calculator 310 may calculate a numerical, mathematical and/or statistical metric or value referred to as “outlier index” for one or more cells. This term is used in the present context in a broad, descriptive, non-restrictive and non-limiting manner to represent a statistical, mathematical and/or numerical value or metric, calculated based at least in part upon the intensities of the pixels that the one or more cells comprise. In this illustrative example, as shown in FIG. 7A, the cells are labeled as 701, 702, 703, 704, 705, 706, 707, 709, 710, 711, 712, 713, 714, 715, 716, 717, 718, 719, 720, 721, 722, 723, 724 and 725.

In a non-limiting, illustrative example, calculator 310 may calculate an outlier index for one or more cells by first calculating a first percentile value of the highest intensities of one or more pixels encompassed by a cell, calculating a second percentile value of the highest intensities of the one or more pixels encompassed by the cell and dividing the first percentile value by the second percentile value to obtain a numerical value for the outlier index.

Furthermore, in a non-limiting, illustrative example, calculator 310 may calculate the 75^{th} percentile of the highest intensities of one or more pixels, calculate the 55^{th} percentile of the highest intensities of the one or more pixels and divide the 75^{th} percentile of the highest intensities by the 55^{th} percentile of the highest intensities to arrive at a numerical value for the outlier index of the cell. However, this need not be so in every embodiment, and as will be appreciated by those of ordinary skill in the art, various other statistical, mathematical and/or numerical values and/or metrics may be calculated based at least in part upon the intensities of one or more pixels. It must be mentioned here that throughout the present context the term “metric” or “metrics” is used in a broad, non-restrictive, non-limiting and descriptive manner to refer to one or more quantities, measures or magnitudes, including but not limited to, percentile, percent, average and/or mean that may be calculated based at least in part upon one or more characteristics of the pixels. Furthermore, in the same or other embodiments, the calculation of one or more metrics including the outlier index for one or more cells may be based at least in part on one or more or any combination of other metrics. Thus the above mentioned statistical, mathematical and/or numerical methods and descriptions for calculating outlier index are illustrative only and should not be interpreted or construed to be limiting or restrictive in any manner whatsoever.

In the illustrative example shown in FIG. 7A, calculator 310 may calculate the outlier index for each one of the 25 cells shown in the figure. Additionally, cells 712 and 721 may be assumed to be optimally aligned with the probe features and may have an outlier index different from all other cells which are not optimally aligned or are misaligned. For example, cells 712 and 721 may have an outlier index, calculated as described above, suitably closer to 1 and the other cells may have an outlier index, also calculated as described above, significantly larger than 1; based on a predefined or user selectable outlier index cells 712 and 721 may be assumed to be optimally aligned and the other cells may be assumed to be misaligned. Grid position adjuster 330 may adjust the misaligned cells around the probe features, for example cells 705, 720 and 724, so that the outlier index of these cells is suitably closer to the outlier index of the optimally aligned cells 712 and 721. Adjuster 330 may move or reposition the grid with respect to the cells in one or more directions by a predefined or user selectable magnitude of movement until a predefined or user selectable outlier index is obtained for the misaligned cells. Additionally, in this illustrative example, adjuster 330 may move a grid or sub grid by a magnitude of movement equal to multiple pixel lengths, including fractional pixel lengths, for example, by 1 pixel length or by 1.2 pixel lengths or 2 pixel lengths and so on. Adjuster 330 may adjust all the cells of the grid that are misaligned by adjusting them around the probe features by employing the method illustratively described above. Additionally, as mentioned earlier, optimal alignment may be based at least in part upon predefined and/or user selectable criteria.

Furthermore, in one embodiment, adjuster 330 may adjust the grid comprising one or more cells around one or more probe features comprising the probe array image, as described above, so that the outlier index of the one or more cells, calculated as described above, is suitably closer to each other or a predefined or a user selectable value.

FIG. 7B is a simplified graphical example of a probe array image after grid placement but before grid alignment, in which the cells having an outlier index different from a predefined or user selectable value are highlighted graphically as brighter elements in the figure. FIG. 7C is a simplified graphical example of the probe array image of FIG. 7B after grid aligner 243 has performed grid alignment, as before, the cells having an outlier index from the predefined or user selectable value are highlighted graphically as brighter elements in the figure.

As will now be appreciated from the illustrative example of FIGS. 7B and 7C, after grid alignment by aligner 243, the number of cells having an outlier index different from the predefined or user selectable value, is reduced in comparison to the number of such cells prior to grid alignment.

It must be noted that adjuster 330 may adjust the position of a grid or sub grid, in one or more directions, by one or more pixels including a fractional number of one or more pixels, as described above. In the new position and/or location of a sub grid, the cells may comprise a fractional or non-whole number of pixels. The fractional or non-whole number of pixels may also be referred to by the descriptive terms ‘sub pixels’ or ‘partial pixels’ in the same or other embodiments.

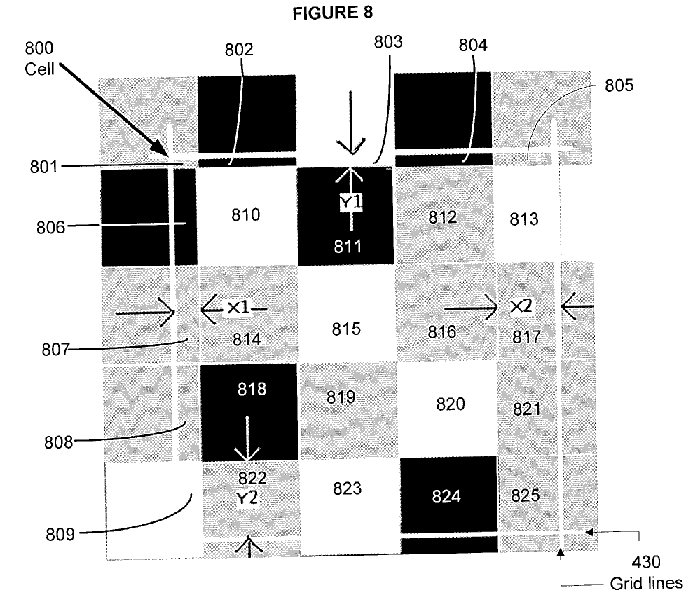

FIG. 8 provides a simplified graphical example of a grid that may be placed over probe features including control features 400A, such that the cells bounded by the grid are comprised of a fractional or non-whole number of pixels. In this illustrative example, cell 800 comprises a plurality of pixels, including complete or whole pixels, for example, the whole pixels 810, 811, 812, 814, 815, 816, 818, 819 and 820. Cell 800 also comprises pixels which are fractions of whole or complete pixels, for example, the fractional pixels 801, 802, 803, 804, 805, 806, 807, 808, 809, 813, 817, 821, 822, 823, 824 and 825. One or more characteristics, including but not limited to the length or height and width of complete or whole pixels may depicted by any numerical value including one or more fractions of the numerical value. It must be mentioned that in the present context the length or height, width and/or other dimensions of the pixels may be represented in any of the units known to those of ordinary skill in the art, including, but not limited to, nanometers, microns, millimeters, picas, inches or meters. In this non-limiting, illustrative example, the length and width of complete or whole pixels 810, 811, 812, 814, 815, 816, 818, 819 and 820 may be equal to a numerical value of 1. The length of fractional pixels 801, 802, 803, 804 and 805 may be represented by a variable ‘y1’, and the width of pixels 802, 803 and 804 may be equal to a numerical value of 1. The width of fractional pixels 801, 806, 807, 808, and 809 may be represented by a variable ‘x1’ and the length of pixels 806, 807 and 808 may be equal to a numerical value of 1. The length of pixels 809, 822, 823, 824 and 825 may be represented by a variable ‘y2’ and the width of pixels 822, 823 and 824 may be equal to a numerical value of 1. The width of pixels 825, 821, 817, 813 and 805 may be represented by a variable ‘x2’ and the length of pixels 821, 817 and 813 may be equal to a numerical value of 1. Furthermore, calculator 310 may calculate a numerical, mathematical and/or statistical metric that may be assigned to each of the pixels of cell 800. In this illustrative example, one such metric, hereafter referred to as ‘weight’ or ‘w’ is calculated and used as described below.

Calculator 310 calculates the weights, w₁, . . . , wₙ, for each of the ‘n’ number of pixels of cell 800. The weights w₁, . . . , wₙ may be represented as a set ‘W’. In this illustrative example, the number of pixels ‘n’, including whole pixels and fractional pixels, is equal to 25, thus the weights of pixels are represented from w₁ to w₂₅ in the set ‘W’ as:

$$W = ( \begin{matrix}{w}_{1} & {w}_{2} & {w}_{3} & {w}_{4} & {w}_{5} \\ {w}_{6} & {w}_{7} & {w}_{8} & {w}_{9} & {w}_{10} \\ {w}_{11} & {w}_{12} & {w}_{13} & {w}_{14} & {w}_{15} \\ {w}_{16} & {w}_{17} & {w}_{18} & {w}_{19} & {w}_{20} \\ {w}_{21} & {w}_{22} & {w}_{23} & {w}_{24} & {w}_{25}\end{matrix} ) = ( \begin{matrix}x1 \cdot y1 & y1 & y1 & y1 & x2 \cdot y1 \\ x1 & 1 & 1 & 1 & x2 \\ x1 & 1 & 1 & 1 & x2 \\ x1 & 1 & 1 & 1 & x2 \\ x1 \cdot y2 & y2 & y2 & y2 & x2 \cdot y2\end{matrix} )$$

In this illustrative example, calculator 310 calculates the weights w₁, . . . , wₙ by multiplying the length of each pixel with the width of the pixel. However this need not be so in every embodiment and in the same or other embodiments one or more weights or ‘w’ may be calculated by employing one or more of other numerical, mathematical and/or statistical methods too numerous to be listed here and well known to those of ordinary skill in the art. Thus the above mentioned statistical, mathematical and/or numerical methods and descriptions for calculating weight or ‘w’ are illustrative only and should not be interpreted or construed to be limiting or restrictive in any manner whatsoever. Furthermore, in the same or other embodiments, the weight or ‘w’ may be calculated by intensity data generator 246.

In one embodiment, aligner 243 may perform a method of local alignment on each corner of each sub grid. Aligner 243 identifies the probe feature or cell located in each corner of the sub grid from the coordinate positions provided by data 236. For each corner cell, aligner 243 searches for bright cells in the surrounding cells that could, for example, include a 5 cell by 5 cell square region with the corner cell being located in the center of the square. Alternatively, aligner 243 may search until a minimum number of bright cells are identified starting with the cells located next to the corner cell and working towards the center of the image. Those of ordinary skill in the art appreciate that aligner 243 may use various numbers of surrounding cells that could occur in a variety of patterns. Aligner 243 identifies all of the bright cells within the surrounding region searched using a predefined or user definable threshold value for brightness.

Final image grid data 225 generated by adjuster 330 may be provided directly to cell intensity data generator 246 and/or stored in probe array data files 140. Final image grid data 225 may comprise of one or more or a combination of grid position co-ordinates, cell position co-ordinates, pixel position co-ordinates, cell intensities, pixel intensities and/or outlier indexes.

In one embodiment, cell intensity data generator 246 generates cell intensity data 226, based at least in part upon final image grid data 225 and/or probe array type data 236. In this illustrative example, one or more cells, for example cell 800, may be comprised of ‘n’ number of pixels, including whole pixels and fractional pixels. Furthermore, the intensity may be represented by a variable ‘v’, such that the intensities of these ‘n’ number of pixels are represented by v₁, . . . , vₙ. Additionally, the weight ‘w’ of each of these ‘n’ numbers of pixels may be represented by w₁, . . . , wₙ. In this illustrative example, the intensities v₁, . . . , vₙ may be further sorted and/or arranged by generator 246, in ascending order as u₁, . . . , uₙ, where u₁≦ . . . ≦uₙ.

Generator 246 may further generate a percentile value, hereafter referred to as ‘P’, of each of the intensity values associated with the pixels of a cell. In this illustrative example, generation of ‘P’ may be based upon the generation of a numerical quantity ‘z’ by generator 246, such that:

z=(n−1).p+1

Where ‘n’ is the number of pixels comprising a cell as described above and ‘p’ is based at least in part upon ‘P’ and, in this illustrative example, may be represented such that:

p=P/100

In this illustrative example, ‘p’ has any numerical value lying between the numbers 0 and 1, including 0 and 1, i.e. 0≦p≦1.

Furthermore, ‘z’ may comprise a non-fractional numerical quantity ‘m’ and fractional numerical quantity ‘f’, such that:

z=m+f

Where, m=floor(z) or the whole number part of the numerical quantity ‘z’; and ‘f’ has any numerical value lying between the numbers 0 and 1, including 0 but not including 1, i.e. 0≦f≦1.

Furthermore, generator 246 may generate ‘P’ based at least in part upon the following equations and/or conditions:

(1.) If f=0 then

P=uₘ

wherein uₘ is the intensity value in the m^{th} position among the intensities u₁, . . . , uₙ described above;

(2). If f>0 then

P=uₘ+f.(uₘ₊₁—uₘ) or

P=(1−f).uₘ+f.uₘ₊₁

wherein uₘ₊₁ is the intensity value in the (m+1)^{th} among the intensities u₁, . . . , uₙ described above.

Cell intensity data 226, may comprise one or more of the data generated above, including, but not limited to, ‘P’ the percentile of intensities of one or more cells comprising the grid placed and/or aligned over a probe array. Furthermore, data 226 may stored in probe array data file 140 for further processing.

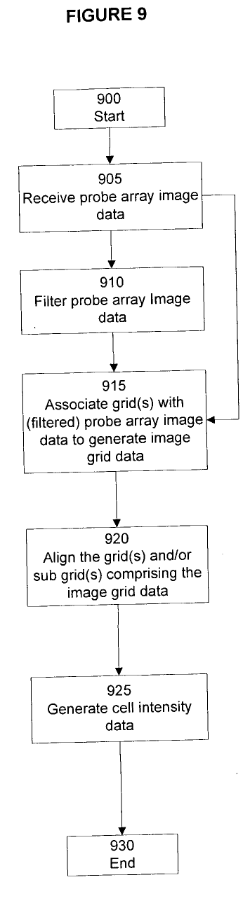

FIG. 9 is a functional block diagram of an embodiment of a method for analysis of probe array images by image processing applications described above. In the illustrated embodiment, the method begins with step 900. In step 905 probe array image data for example, image data 222 is received for further processing. The step of receiving data may be performed, for example, by applications 199A. The data so received may be filtered before further processing as shown in step 910 by a filtering application, for example, raw image filter 240 as described above. In step 915 one or more grids may be associated with the data received in step 905 or with data provided after step 910, to generate image grid data, by an application such as grid associater 242 as described above. In step 920, grid aligner 243 may adjust one or more grids/sub grids for optimal alignment with one or more features that the probe array image data comprises. In step 925 cell intensity data generator 246 calculates the intensities of one or more cells of the grid and generates cell intensity data as described above. Finally, step 930 signifies the end of the method.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

Also, the functions of several elements may, in alternative embodiments, be carried out by fewer, or a single, element. Similarly, in some embodiments, any functional element may perform fewer, or different, operations than those described with respect to the illustrated embodiment. Also, functional elements shown as distinct for purposes of illustration may be incorporated within other functional elements in a particular implementation. Also, the sequencing of functions or portions of functions generally may be altered. Certain functional elements, files, data structures, and so on may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. A system, comprising:
a grid associater constructed and arranged to associate one or more grids with a probe array image based, at least in part, upon a positional placement of at least one of one or more control features;
a grid aligner constructed and arranged to align at least one of the one or more grids with one or more pixels of the one or more control features based, at least in part, upon a metric value determined by one or more characteristics of the one or more pixels; and
a cell intensity data generator constructed and arranged to generate one or more cell intensity values.

2. The system of claim 1, wherein:
the probe array image includes an image of a synthesized probe array or a spotted probe array.

3. The system of claim 1, wherein:
the probe array image includes a filtered probe array image.

4. The system of claim 1, wherein:
the one or more control features comprise one or more probe sets.

5. The system of claim 1, wherein:
the one or more control features comprise one or more chrome features.

6. The system of claim 1, wherein:
each of the one or more cell intensity values is based, at least in part, upon one or more pixel intensity values associated with an area defined by one of the one or more grids.

7. The system of claim 1, wherein:
the metric value includes an average intensity value, wherein the one or more characteristics includes a pixel intensity value.

8. The system of claim 1, wherein:
the metric value is compared to a predefined value or user selected value.

9. The system of claim 1, wherein:
the alignment is based, at least in part, upon the similarity between the metric value and the predefined or user selected value.

10. A method, comprising the acts of:
associating one or more grids with a probe array image based, at least in part, upon a positional placement of at least one of one or more control features;
aligning at least one of the one or more grids with one or more pixels of the one or more control features based, at least in part, upon a metric value determined by one or more characteristics of the one or more pixels; and
generating one or more cell intensity values.

11. The method of claim 10, wherein:
the probe array image includes an image of a synthesized probe array or a spotted probe array.

12. The method of claim 10, wherein:
the probe array image includes a filtered probe array image.

13. The method of claim 10, wherein:
the one or more control features comprise one or more probe sets.

14. The method of claim 10, wherein:
the one or more control features comprise one or more chrome features.

15. The method of claim 10, wherein:
each of the one or more cell intensity values is based, at least in part, upon one or more pixel intensity values associated with an area defined by one of the one or more grids.

16. The method of claim 10, wherein:
the metric value includes an average intensity value, wherein the one or more characteristics include a pixel intensity value.

17. The method of claim 10, wherein:
the metric value is compared to a predefined value or a user selected value.

18. The method of claim 17, wherein:
the act of aligning is based, at least in part, upon the similarity between the metric value and the predefined or user selected value.

19. A system for probe array image analysis, comprising:
a grid associater constructed and arranged to associate one or more grids with a probe array image based, at least in part, upon a positional placement of at least one of one or more control features;
a grid data calculator constructed and arranged to determine a ratio of a first set of pixel intensity values and second set of pixel intensity values associated with each area of a plurality of areas defined by one of the one or more grids; and
a grid position adjuster constructed and arranged to adjust the positional association of at least one of the one or more grids based, at least in part upon the determined ratio.

20. The system of claim 16, wherein:
the one or more probe arrays comprise synthesized probe arrays or spotted probe arrays.

21. The system of claim 16, wherein:
the one or more control features comprise one or more probe sets.

22. A method, comprising the acts of:
associating one or more grids with a probe array image based, at least in part, upon a positional placement of at least one of one or more control features;
determining a ratio of a first set of pixel intensity values and second set of pixel intensity values associated with each area of a plurality of areas defined by one of the one or more grids; and
adjusting the positional association of at least one of the one or more grids based, at least in part upon the determined ratio.

23. The method of claim 29, wherein:
the one or more probe arrays comprise synthesized probe arrays or spotted probe arrays.

24. The method of claim 16, wherein:
the one or more control features comprise one or more probe sets.

25. A system for probe array image analysis, comprising:
a grid associater constructed and arranged to associate one or more grids with a probe array image based, at least in part, upon a positional placement of at least one of one or more control features; and
a cell intensity data generator constructed and arranged to generate one or more cell intensity values, wherein each cell intensity value is based, at least in part, upon one or more weighted pixel intensity values associated with an area defined by one of the one or more grids.

26. The system of claim 25, wherein:
the one or more probe arrays comprise synthesized probe arrays or spotted probe arrays.

27. The system of claim 25, wherein:
the weighted pixel intensity value associated with a pixel is based, at least in part, upon the length and width of the pixel.

28. A method for probe array image analysis, comprising:
associating one or more grids with a probe array image based, at least in part, upon a positional placement of at least one of one or more control features; and
generating one or more cell intensity values, wherein each cell intensity value is based, at least in part, upon one or more weighted pixel intensity values associated with an area defined by one of the one or more grids.

29. The method of claim 28, wherein:
the one or more probe arrays comprise synthesized probe arrays or spotted probe arrays.

30. The method of claim 28, wherein:
the weighted pixel intensity value associated with a pixel is based, at least in part, upon the length and width of the pixel.
